(19) United States
(12) Patent Application Publication
(10) Pub. No.: US 2017/0258304 A1
(43) Pub. Date: Sep. 14, 2017

(54) IMAGING DEVICE, ENDOSCOPE, AND CAPSULE ENDOSCOPE

(71) Applicant: OLYMPUS CORPORATION, Tokyo (JP)

(72) Inventors: Ken IOKA, Tokyo (JP); Kazunori YOSHIZAKI, Tokyo (JP); Sunao KIKUCHI, Tokyo (JP); Yasuhiro KOMIYA, Sagamihara-shi (JP); Yasuhiro FUKUNAGA, Tokyo (JP)

(73) Assignee: OLYMPUS CORPORATION, Tokyo (JP)

(21) Appl. No.: 15/607,485

(22) Filed: May 27, 2017

Related U.S. Application Data
(63) Continuation of application No. PCT/JP2014/082316, filed on Dec. 5, 2014.

(51) Int. Cl. A61B 1/04; A61B 1/06; A61B 1/05; G02B 23/24

(52) U.S. Cl. CPC A61B 1/041; G02B 23/2415; A61B 1/06; A61B 1/05; A61B 1/00004

(57) ABSTRACT

An imaging device includes an image sensor. The image sensor includes: a light receiving unit having pixels configured to receive light and generate an imaging signal according to an amount of the received light; a color filter having a filter unit disposed corresponding to the pixels, the filter unit including first band filters for passing light of a wavelength band of a primary color or a complementary color and including at least one second band filter for passing narrow-band light whose wavelength band is narrower than the wavelength band of the light passing through each of the first band filters; and an output unit configured to output the imaging signal under conditions that an amount of light incident on a second pixel corresponding to the at least one second band filter is greater than an amount of light incident on each of first pixels corresponding to the first band filters.

### CROSS REFERENCES TO RELATED APPLICATIONS

This application is a continuation of PCT international application Ser. No. PCT/JP2014/082316 filed on Dec. 5, 2014 which designates the United States, incorporated herein by reference.

### BACKGROUND

1. Technical Field

The disclosure relates to an imaging device, an endoscope, and a capsule endoscope that are configured to be introduced into a subject to capture images of a body cavity of the subject.

2. Related Art

In recent years, regarding the endoscope, there is a known technique in which a filter unit where a plurality of wide-band filters having wide-band wavelength transmission characteristics in a visible region and a plurality of narrow-band filters having narrow-band wavelength transmission characteristics are arranged in a grid pattern is provided to an image sensor and thereby a narrow-band image of a blue region, where tissues located in a deep position from a surface of living tissues can be clearly observed, and a normal color wide-band image are obtained at the same time (see JP 2007-54113 A).

### SUMMARY

In some embodiments, an imaging device includes an image sensor. The image sensor includes: a light receiving unit having a plurality of pixels arranged two-dimensionally, the plurality of pixels being configured to receive light from outside and generate an imaging signal in accordance with an amount of the received light; a color filter having a filter unit disposed corresponding to the plurality of pixels, the filter unit including a plurality of first band filters for passing light of a wavelength band of a primary color or a complementary color and including at least one second band filter for passing narrow-band light whose wavelength band is narrower than the wavelength band of the light passing through each of the plurality of first band filters; and an output unit configured to output the imaging signal generated by the light receiving unit under conditions that an amount of light incident on a second pixel of the plurality of pixels corresponding to the at least one second band filter is greater than an amount of light incident on each of first pixels of the plurality of pixels corresponding to the plurality of first band filters.

In some embodiments, an endoscope includes an insertion portion. The insertion portion has the imaging device at a distal end of the insertion portion.

In some embodiments, a capsule endoscope includes a capsule-shaped casing configured to be inserted into a subject, and the imaging device provided inside the capsule-shaped casing.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating a schematic configuration of a capsule endoscope system according to a first embodiment of the present invention;

FIG. 2 is a block diagram illustrating a functional configuration of a capsule endoscope according to the first embodiment of the present invention;

FIG. 3 is a diagram schematically illustrating a configuration of a color filter according to the first embodiment of the present invention;

FIG. 4 is a flowchart illustrating an overview of processing performed by the capsule endoscope according to the first embodiment of the present invention;

FIG. 5 is a cross-sectional view schematically illustrating a configuration of an image sensor according to a second embodiment of the present invention;

FIG. 6 is a cross-sectional view schematically illustrating a configuration of an image sensor according to a modified example of the second embodiment of the present invention;

FIG. 7 is a block diagram illustrating a functional configuration of a capsule endoscope according to a third embodiment of the present invention;

FIG. 8 is a diagram illustrating a relationship between transmittance and wavelength of each filter included in a color filter according to the third embodiment of the present invention;

FIG. 9 is a diagram illustrating a relationship between transmittance and wavelength of an optical filter according to the third embodiment of the present invention;

FIG. 10 is a diagram illustrating a relationship between transmittance and wavelength of a combination of the color filter and the optical filter according to the third embodiment of the present invention;

FIG. 11 is a schematic diagram of an optical filter according to a modified example of the third embodiment of the present invention; and

FIG. 12 is a diagram schematically illustrating an arrangement of the optical filter according to the modified example of the third embodiment of the present invention.

### DETAILED DESCRIPTION

Exemplary embodiments for carrying out the present invention will be described below in detail with reference to the drawings. The present invention is not limited by the embodiments described below. Each drawing referred to in the description below merely schematically illustrates shapes, sizes, and positional relationships in a degree such that contents of the present invention can be understood. Therefore, the present invention is not limited to the sizes, the shapes, and the positional relationships illustrated in each drawing. In the description below, reference will be made to an exemplary capsule endoscope system which includes a processing device for receiving a wireless signal from a capsule endoscope, which is configured to be introduced into a subject to capture in-vivo images of the subject, and displaying the in-vivo images of the subject. However, the present invention is not limited by this embodiments. The same reference numerals are used to designate the same elements throughout the drawings.

### First Embodiment

### Schematic Configuration of Capsule Endoscope System

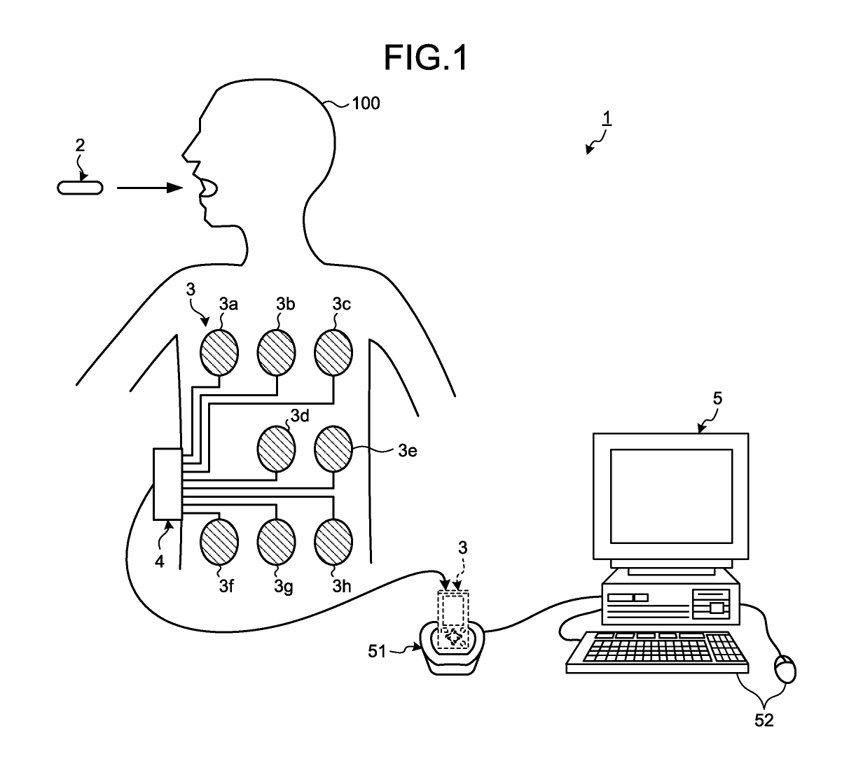

FIG. 1 is a schematic diagram illustrating a simplified configuration of a capsule endoscope system according to a first embodiment of the present invention.

A capsule endoscope system 1 illustrated in FIG. 1 includes a capsule endoscope 2 that captures in-vivo images in a subject 100, a receiving antenna unit 3 that receives a wireless signal transmitted from the capsule endoscope 2 introduced into the subject 100, a receiving device 4 to which the receiving antenna unit 3 is detachably connected and which performs predetermined processing on the wireless signal received by the receiving antenna unit 3 to record or display the wireless signal, and an image processing device 5 that processes and/or displays an image corresponding to image data inside the subject 100, which is captured by the capsule endoscope 2.

The capsule endoscope 2 has an imaging function for capturing images inside the subject 100 and a wireless communication function for transmitting in-vivo information including image data obtained by capturing images inside the subject 100 to the receiving antenna unit 3. After the capsule endoscope 2 is swallowed into the subject 100, the capsule endoscope 2 passes through the esophagus inside the subject 100 and moves inside a body cavity of the subject 100 by a peristaltic movement of a digestive tract lumen. While moving inside the body cavity of the subject 100, the capsule endoscope 2 sequentially captures images inside the body cavity of the subject 100 at a minute time interval, for example, at 0.5 sec intervals (2 fps), generates image data of images captured inside the subject 100, and sequentially transmits the image data to the receiving antenna unit 3. The detailed configuration of the capsule endoscope 2 will be described later.

The receiving antenna unit 3 includes receiving antennas 3a to 3h. The receiving antennas 3a to 3h receive the wireless signal from the capsule endoscope 2 and transmit the wireless signal to the receiving device 4. The receiving antennas 3a to 3h are configured to include loop antennas. The receiving antennas 3a to 3h are arranged at predetermined positions on the external surface of the subject 100, for example, at positions corresponding to each organ in the subject 100 which is a passing route of the capsule endoscope 2.

The receiving device 4 records image data inside the subject 100 included in the wireless signal transmitted from the capsule endoscope 2 through the receiving antennas 3a to 3h, or displays an image corresponding to the image data inside the subject 100. The receiving device 4 records position information of the capsule endoscope 2 and time information indicating time in association with the image data received through the receiving antennas 3a to 3h. The receiving device 4 is housed in a receiving device holder (not illustrated in the drawings) and carried by the subject 100 while examination by the capsule endoscope 2 is being performed, that is, for example, from when the capsule endoscope 2 is introduced from the mouth of the subject 100 to when the capsule endoscope 2 passes through the digestive tract and is discharged from the subject 100. After the examination by the capsule endoscope 2 is completed, the receiving device 4 is removed from the subject 100 and connected to the image processing device 5 to transmit image data and the like received from the capsule endoscope 2.

The image processing device 5 displays an image corresponding to the image data inside the subject 100 received through the receiving device 4. The image processing device 5 includes a cradle 51 that reads image data and the like from the receiving device 4 and an operation input device 52 such as a keyboard and a mouse. The cradle 51 acquires from the receiving device 4 image data, position information and time information associated with the image data, and related information such as identification information of the capsule endoscope 2 when the receiving device 4 is attached, and transmits the acquired various information to the image processing device 5. The operation input device 52 receives an input from a user. The user diagnoses the subject 100 by observing living body regions such as esophagus, stomach, small intestine, and large intestine inside the subject 100 while operating the operation input device 52 and seeing images inside the subject 100 sequentially displayed by the image processing device 5.

Configuration of Capsule Endoscope

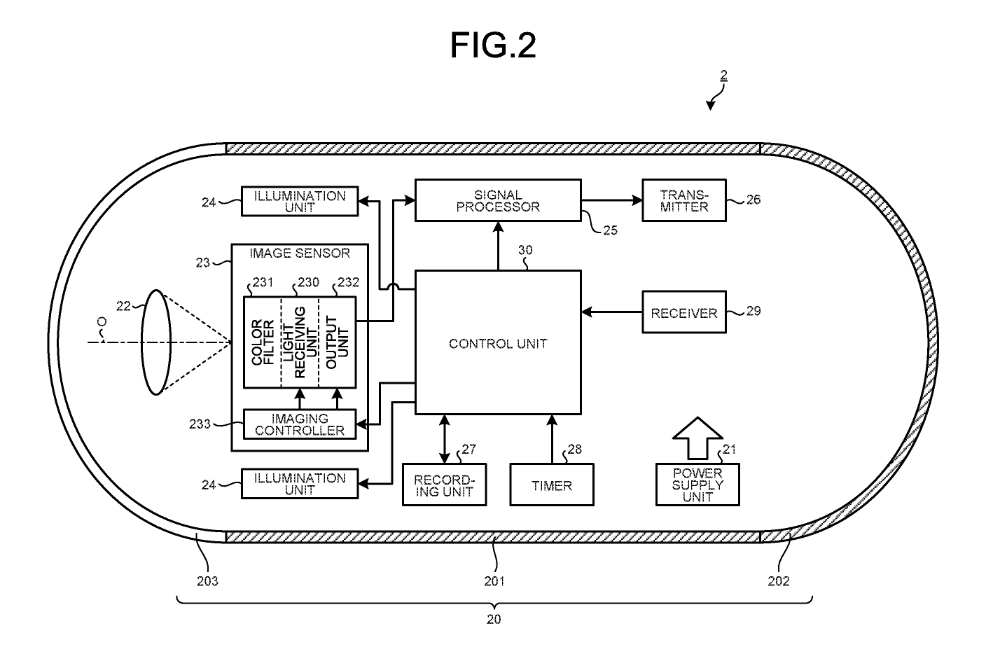

Next, a detailed configuration of the capsule endoscope 2 described in FIG. 1 will be described. FIG. 2 is a block diagram illustrating a functional configuration of the capsule endoscope 2. The capsule endoscope 2 illustrated in FIG. 2 has a casing 20, a power supply unit 21, an optical system 22, an image sensor 23, an illumination unit 24, a signal processor 25, a transmitter 26, a recording unit 27, a timer 28, a receiver 29, and a control unit 30.

The casing 20 has a capsule shape and is small enough to easily inserted into the subject 100. The casing 20 has a tubular tube portion 201 and dome-shaped dome portions 202 and 203 that close open ends of both sides of the tube portion 201. The tube portion 201 and the dome portion 202 are formed by using an opaque colored member that blocks visible light. The dome portion 203 is formed by using an optical member that can transmit predetermined wavelength band light such as visible light. As illustrated in FIG. 2, the casing 20 formed by the tube portion 201 and the dome portions 202 and 203 houses the power supply unit 21, the optical system 22, the image sensor 23, the illumination unit 24, the signal processor 25, the transmitter 26, the recording unit 27, the timer 28, the receiver 29, and the control unit 30.

The power supply unit 21 supplies power to each unit of the capsule endoscope 2. The power supply unit 21 includes a primary battery or a secondary battery such as a button battery and a power supply circuit that raises a voltage supplied from the button battery. The power supply unit 21 has a magnetic switch and switches on/off of power supply by a magnetic field applied from outside.

The optical system 22 includes a plurality of lenses to collect reflection light of illumination light emitted by the illumination unit 24 onto an imaging surface of the image sensor 23 to form an object image. The optical system 22 is arranged inside the casing 20 such that the optical axis of the optical system 22 corresponds to a central axis O in the longitudinal direction of the casing 20.

The image sensor 23 receives the object image formed on a light receiving surface by the optical system 22 and performs photoelectric conversion on the object image to generate an imaging signal (image data) of the subject 100 under control of the control unit 30. Specifically, the image sensor 23 generates the imaging signal of the subject 100 by capturing images of the subject 100 at a reference frame rate, for example, at a frame rate of 4 fps under control of the control unit 30. Examples of the image sensor 23 include as complementary metal oxide semiconductor (CMOS).

The image sensor 23 has a light receiving unit 230 having a plurality of pixels that is two-dimensionally arranged, receives light from outside, and generates and outputs an imaging signal in accordance with the amount of received light, a color filter 231 in which a filter unit including a plurality of first band filters (hereinafter referred to as “wide-band filters”) for passing light of a wavelength band of a primary color or a complementary color and a second band filter (hereinafter referred to as a “narrow-band filter”) for passing light of a wavelength band narrower than that of each of the plurality of first band filters is arranged in association with the plurality of pixels, an output unit 232 that outputs an imaging signal generated by the light receiving unit 230 when the amount of light greater than that of incident on pixels corresponding to the wide-band filters is incident on pixels corresponding to the narrow-band filter, and an imaging controller 233 that reads a second imaging signal (hereinafter referred to as a “narrow-band image signal”) from the pixels corresponding to the narrow-band filter (hereinafter referred to as “narrow-band pixels”) after reading a first imaging signal (hereinafter referred to as a “wide-band image signal”) from the pixels corresponding to the wide-band filters (hereinafter referred to as “wide-band pixels”) in the light receiving unit 230.

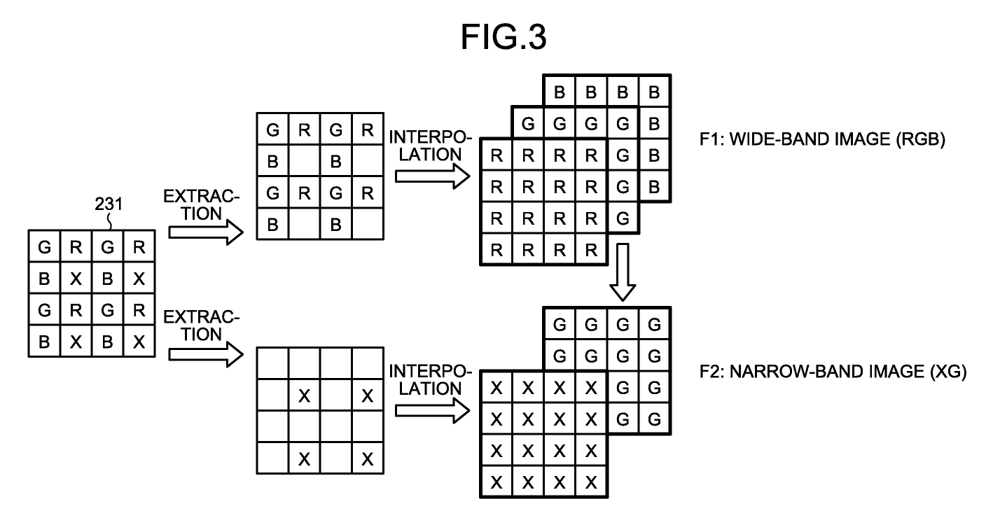

FIG. 3 is a diagram schematically illustrating a configuration of the color filter 231 and generation of the wide-band image and the narrow-band image. As illustrated in FIG. 3, the color filter 231 includes a wide-band filter R for passing a red color component, a wide-band filter G for passing a green color component, a wide-band filter B for passing a blue color component, and a narrow-band filter X for passing a wavelength band of 415 nm±30 nm. Predetermined image processing (for example, interpolation such as demosaicing) is performed on the imaging signal, which is generated by each pixel of the light receiving unit 230 by using the color filter 231 configured as described above, by any one of the signal processor 25, the receiving device 4, and the image processing device 5, and thereby a wide-band image F1 is generated from wide-band R, G, and B pixel signals and a narrow-band image F2 is generated from narrow-band X pixel signals and wide-band G pixel signals.

The illumination unit 24 irradiates an object with light in an imaging visual field of the image sensor 23 in synchronization with a frame rate of the image sensor 23 under control of the control unit 30. The illumination unit 24 includes a light emitting diode (LED), a drive circuit, and the like.

The signal processor 25 performs predetermined image processing on the imaging signal input from the image sensor 23 and outputs the imaging signal to the transmitter 26. Here, the predetermined image processing is noise reduction, gain-up, demosaicing, and the like. Further, the signal processor 25 generates a wide-band image (see the wide-band image F1 in FIG. 3) based on a wide-band image signal included in the imaging signal output from the output unit 232 of the image sensor 23, generates a narrow-band image (see the narrow-band image F2 in FIG. 3) based on a wide-band image signal output from wide-band pixels corresponding to the wide-band filter G and a narrow-band image signal output from narrow-band pixels corresponding to the narrow-band filter X, and transmits the wide-band image and the narrow-band image to the transmitter 26.

The transmitter 26 wirelessly transmits the wide-band image and the narrow-band image sequentially input from the signal processor 25 to the outside. The transmitter 26 includes a transmitting antenna and a modulation circuit that modulates the wide-band image or the narrow-band image into a wireless signal by performing signal processing such as modulation on the wide-band image or the narrow-band image.

The recording unit 27 stores a program for various operations performed by the capsule endoscope 2 and identification information for identifying the capsule endoscope 2.

The timer 28 has a clocking function. The timer 28 outputs clock data to the control unit 30.

The receiver 29 receives a wireless signal transmitted from outside and outputs the wireless signal to the control unit 30. The receiver 29 includes a receiving antenna and a demodulation circuit for performing signal processing, such as demodulation, on the wireless signal and then outputting the wireless signal to the control unit 30.

The control unit 30 controls operations of each unit of the capsule endoscope 2. The control unit 30 causes the illumination unit 24 to emit light. Further, the control unit 30 causes the image sensor 23 to capture an image and generate an imaging signal in synchronization with the irradiation timing of the illumination unit 24. The control unit 30 includes a central processing unit (CPU).

The capsule endoscope 2 configured as described above sequentially captures images inside a body cavity of the subject 100 at a minute time interval while moving inside the body cavity of the subject 100, generates image data corresponding to an imaging signal of images captured inside the subject 100, and sequentially transmits the image data to the receiving antenna unit 3.

Processing of Capsule Endoscope

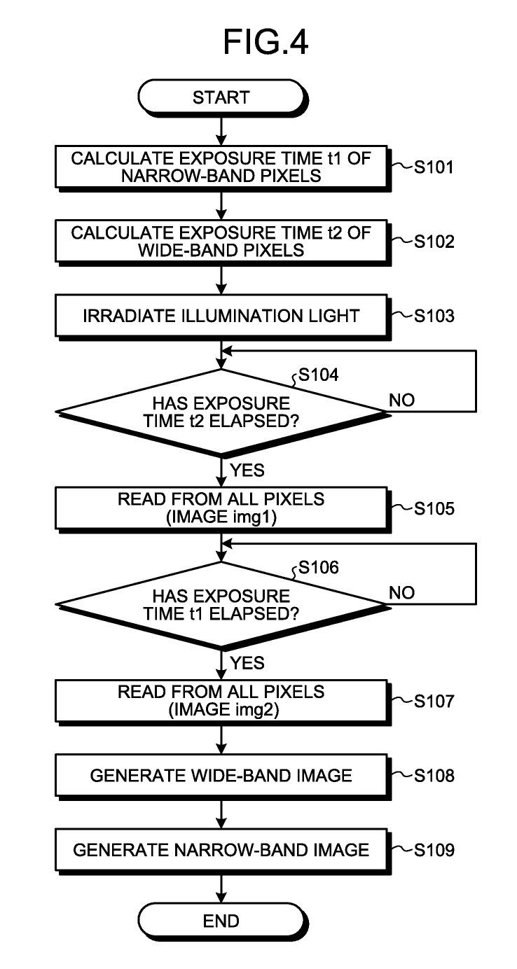

Next, processing performed by the capsule endoscope 2 will be described. FIG. 4 is a flowchart illustrating an overview of the processing performed by the capsule endoscope 2. FIG. 4 illustrates processing performed by the capsule endoscope 2 in a single capturing operation.

As illustrated in FIG. 4, first, the imaging controller 233 calculates exposure time t1 of the narrow-band pixels based on the sensitivity of the narrow-band filter X and the amount of light emitted by the illumination unit 24 (step S101) and calculates exposure time t2 of the wide-band pixels based on the sensitivity of the wide-band filters R, G, and B and the amount of light emitted by the illumination unit 24 (step S102). Here, the sensitivity of the narrow-band pixels is lower than that of the wide-band pixels, and therefore the exposure time t1 of the narrow-band pixels is longer than the exposure time t2 Of the wide-band pixels (t1>t2). Thereafter, the control unit 30 causes the illumination unit 24 to emit illumination light (step S103).

Subsequently, when the exposure time t2 has elapsed (step S104: Yes), the imaging controller 233 performs non-destructive reading of the imaging signal from all the pixels of the light receiving unit 230 (step S105). In this case, the output unit 232 outputs an image (hereinafter referred to as an “image img1”) corresponding to the imaging signals read from all the pixels of the light receiving unit 230 to the signal processor 25. After step S105, the capsule endoscope 2 proceeds to step S106 described later. On the other hand, when the exposure time t2 has not yet elapsed (step S104: No), the capsule endoscope 2 does not perform the non-destructive reading until the exposure time t2 has elapsed.

In step S106, when the exposure time t1 has elapsed (step S106: Yes), the imaging controller 233 reads the imaging signal from all the pixels of the light receiving unit 230 (step S107). In this case, the output unit 232 outputs an image (hereinafter referred to as an “image img2”) corresponding to the imaging signals read from all the pixels of the light receiving unit 230 to the signal processor 25. At this time, the imaging controller 233 performs reset processing on all the pixels of the light receiving unit 230 to initialize the charges of all the pixels. After step S107, the capsule endoscope 2 proceeds to step S108 described later. On the other hand, when the exposure time t1 has not yet elapsed (step S106: No), the capsule endoscope 2 does not perform reading until the exposure time t1 has elapsed.

In step S108, the signal processor 25 generates a color wide-band image based on the image img1 output from the image sensor 23. Specifically, the signal processor 25 generates the wide-band image by using wide-band signals read from the wide-band pixels (pixels corresponding to the wide-band filters R, G, and B) included in the image img1.

Subsequently, the signal processor 25 generates a narrow-band image based on the image img2 output from the image sensor 23 (step S109). Specifically, the signal processor 25 generates the narrow-band image by using a wide-band signal (G component) read from a wide-band image corresponding to the wide-band filter G included in the image img1 and a narrow-band signal read from narrow-band pixels corresponding to the narrow-band filter X included in the image img2. Thereby, even when a wide-band image and a narrow-band image are captured at the same time, each of the wide-band image and the narrow-band image can be acquired with high image quality.

According to the first embodiment described above, the imaging controller 233 reads the wide-band signals from the wide-band pixels corresponding to the wide-band filters and thereafter reads a narrow-band signal from the narrow-band pixels corresponding to the narrow-band filter, and then the output unit 232 outputs the wide-band signals and the narrow-band signal as the imaging signals. Therefore, even when a wide-band image and a narrow-band image are captured at the same time, a high-quality narrow-band image can be acquired.

Further, according to the first embodiment, it is possible to acquire signals of the wide-band pixels and a signal of the narrow-band pixels almost at the same time, thereby to obtain an image in which a position deviation between the wide-band pixels and the narrow-band pixels is suppressed to minimum. Therefore, when the wide-band image and the narrow-band image generated from the wide-band pixels and the narrow-band pixels are superimposed, it is possible to omit image processing for positioning of the images.

Further, according to the first embodiment, it is possible to acquire the wide-band image and the narrow-band image by using only the illumination unit 24 for emitting normal white light, thereby to achieve a small-sized capsule endoscope 2.

### Second Embodiment

Next, a second embodiment of the present invention will be described. In a capsule endoscope system according to the second embodiment, a configuration of an image sensor of a capsule endoscope is different from the configuration of the image sensor 23 of the capsule endoscope 2 according to the first embodiment described above. Therefore, in the description below, the configuration of the image sensor of the capsule endoscope according to the second embodiment will be described. The same elements as those in the capsule endoscope according to the first embodiment are denoted by the same reference numerals and the explanation thereof will be omitted.

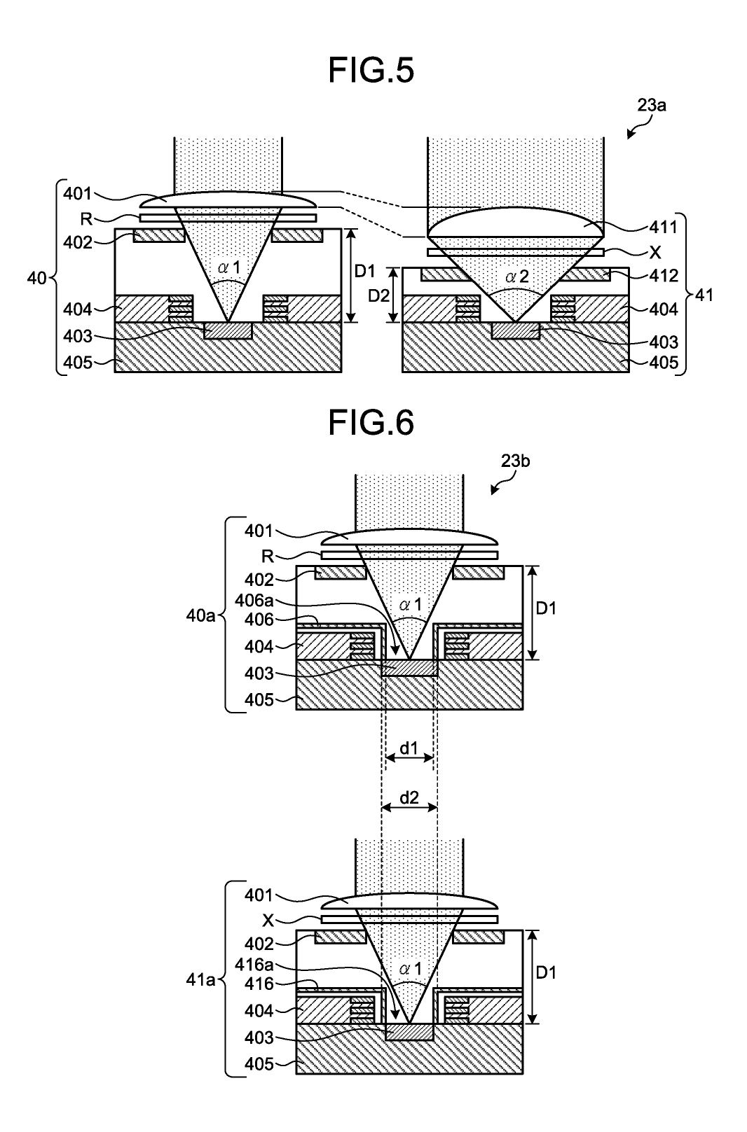

FIG. 5 is a cross-sectional view schematically illustrating a configuration of the image sensor according to the second embodiment. In FIG. 5, regarding a plurality of pixel units included in the image sensor, a wide-band pixel unit corresponding to one wide-band filter and a narrow-band pixel unit corresponding to one narrow-band filter will be described.

An image sensor 23a illustrated in FIG. 5 has a wide-band pixel unit 40 and a narrow-band pixel unit 41.

The wide-band pixel unit 40 has at least a first microlens 401 that collects light, a wide-band filter R, a light blocking layer 402 that blocks a part of the light collected by the first microlens 401, a photodiode 403 as a pixel that receives the light collected by the first microlens 401, a wiring layer 404 where various wirings are laminated, and a silicon substrate 405 where the photodiode 403 is formed. The wide-band pixel unit 40 is formed by laminating the silicon substrate 405, the wiring layer 404, the photodiode 403, the light blocking layer 402, and the wide-band filter R, and the first microlens 401 in this order.

The narrow-band pixel unit 41 has at least a second microlens 411 that collects light, a narrow-band filter X, a light blocking layer 412, the photodiode 403, the wiring layer 404, and the silicon substrate 405. The narrow-band pixel unit 41 is formed by laminating the silicon substrate 405, the wiring layer 404, the photodiode 403, the light blocking layer 412, and the narrow-band filter X, and the second microlens 411 in this order. A viewing angle α2 of the second microlens 411 is made greater than a viewing angle α1 of the first microlens 401 (α2>α1). Further, regarding the narrow-band pixel unit 41, the viewing angle α2 of the second microlens 411 is greater than the viewing angle α1 of the first microlens 401, and therefore a thickness D2 of the light blocking layer 412 can be smaller than a thickness D1 of the light blocking layer 402.

The image sensor 23a includes the wide-band pixel unit 40 and the narrow-band pixel unit 41 as described above, and as an optical member that makes the amount of light incident on the wide-band pixel unit 40 smaller than the amount of light incident on the narrow-band pixel unit 41, the viewing angle α2 of the second microlens 411 is made greater than the viewing angle α1 of the first microlens 401. With this structure, the image sensor 23a can make the amount of light incident on the narrow-band pixel greater than the amount of light incident on the wide-band pixel, and therefore even when the narrow-band image and the wide-band image are captured at the same time, each of the narrow-band image and the wide-band image can be acquired with high image quality.

According to the second embodiment described above, the viewing angle α2 of the second microlens 411 is made greater than the viewing angle α1 of the first microlens 401, and therefore the amount of light incident on the narrow-band pixel unit 41 can be greater than the amount of light incident on the wide-band pixel unit 40. Therefore, even when the narrow-band image and the wide-band image are captured, it is possible to acquire a high-quality narrow-band image.

In the second embodiment, a distance between the first microlens 401 and the second microlens 411 over the color filter 231 may be changed. For example, when the first microlens 401 and another first microlens 401 are adjacent to each other, a certain distance is provided between the first microlens 401 and the other first microlens 401, and when the first microlens 401 and the second microlens 411 are adjacent to each other, no gap is provided between the first microlens 401 and the second microlens 411, and the first microlens 401 and the second microlens 411 may be provided over the color filter 231.

### Modified Example of Second Embodiment

Next, a modified example of the second embodiment will be described. FIG. 6 is a cross-sectional view schematically illustrating a configuration of an image sensor according to the modified example of the second embodiment. In FIG. 6, regarding a plurality of pixel units included in the image sensor, a wide-band pixel unit corresponding to one wide-band filter and a narrow-band pixel unit corresponding to one narrow-band filter will be described.

As illustrated in FIG. 6, an image sensor 23b has a wide-band pixel unit 40a and a narrow-band pixel unit 41a.

The wide-band pixel unit 40a has a first light blocking film 406 as an optical member in addition to a configuration of the wide-band pixel unit 40 according to the second embodiment described above. The first light blocking film 406 is arranged between the wide-band filter R and the photodiode 403 and has a first aperture portion 406a where an aperture d1 of a predetermined size is formed.

The narrow-band pixel unit 41a has a second light blocking film 416 as an optical member in addition to a configuration of the narrow-band pixel unit 41 according to the second embodiment described above. The second light blocking film 416 is arranged between the narrow-band filter X and the photodiode 403 and has a second aperture portion 416a where an aperture d2 larger than the first aperture portion 406a is formed (d2<d1).

The image sensor 23b includes the wide-band pixel unit 40a and the narrow-band pixel unit 41a as described above, and as an optical member that makes the amount of light incident on the wide-band pixel unit 40a smaller than the amount of light incident on the narrow-band pixel unit 41a, the aperture d2 of the second aperture portion 416a is made greater than the aperture d1 of the first aperture portion 406a. With this structure, the image sensor 23b can make the amount of light incident on the narrow-band pixel greater than the amount of light incident on the wide-band pixel, and therefore even when the narrow-band image and the wide-band image are captured at the same time, a high-quality narrow-band image can be acquired.

According to the modified example of the second embodiment, the aperture d2 of the second aperture portion 416a is made greater than the aperture d1 of the first aperture portion 406a, and therefore the amount of light incident on the narrow-band pixel unit 41a can be greater than the amount of light incident on the wide-band pixel unit 40a. Therefore, even when the narrow-band image and the wide-band image are captured, each of the narrow-band image and the wide-band image can be acquired with high image quality.

In the modified example of the second embodiment, the amount of light incident on the photodiode 403 is adjusted by changing the size of the aperture of the first aperture portion 406a of the first light blocking film 406 of the wide-band pixel unit 40a and the size of the aperture of the second aperture portion 416a of the second light blocking film 416 of the narrow-band pixel unit 41a. However, the amount of light incident on the photodiode 403 may be adjusted by changing the area and the size of the wiring layer 404 formed in the color filter 231 and the photodiode 403 for each of the wide-band pixel unit 40a and the narrow-band pixel unit 41a.

### Third Embodiment

Next, a third embodiment of the present invention will be described. In a capsule endoscope system according to the third embodiment, a configuration of a capsule endoscope is different from the configuration of the capsule endoscope according to the first embodiment described above. Therefore, in the description below, the configuration of the capsule endoscope according to the third embodiment will be described. In the description below, the same elements as those in the capsule endoscope 2 according to the first embodiment are denoted by the same reference numerals and the explanation thereof will be omitted.

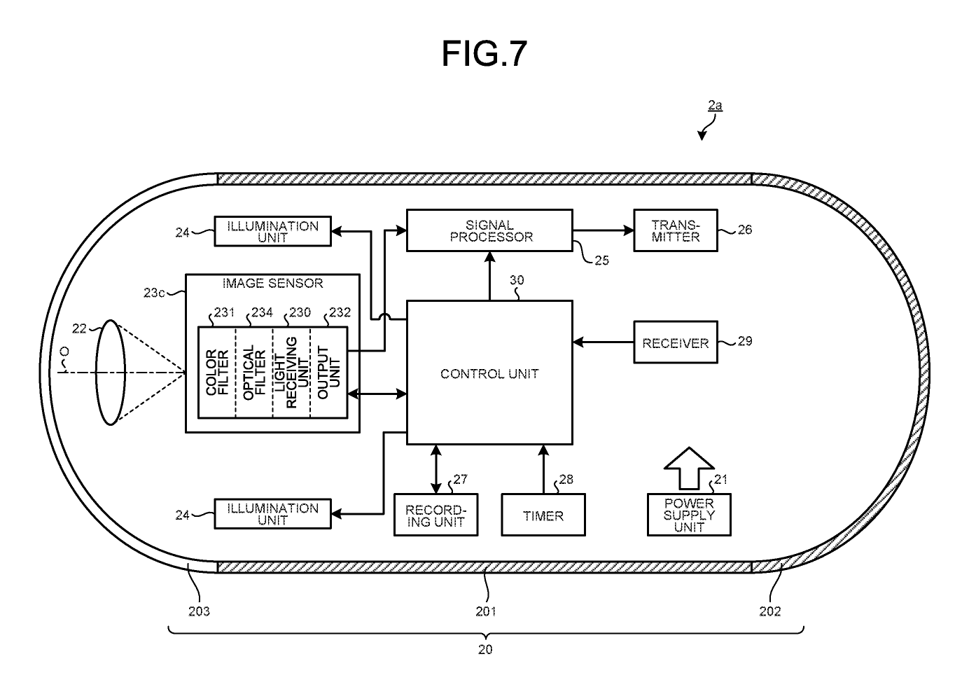

FIG. 7 is a block diagram illustrating a functional configuration of a capsule endoscope 2a according to the third embodiment. The capsule endoscope 2a illustrated in FIG. 7 includes an image sensor 23c instead or the image sensor 23 of the capsule endoscope 2 according to the first embodiment described above.

The image sensor 23c generates an imaging signal (image data) of a subject 100 by receiving an object image formed on a light receiving surface by an optical system 22 and performing photoelectric conversion under control of the control unit 30. The image sensor 23c has a light receiving unit 230, a color filter 231, an output unit 232, and an optical filter 234.

The optical filter 234 includes a low-pass filter for passing at least narrow-band light and is arranged between the color filter 231 and the light receiving unit 230. The optical filter 234 has a rectangular shape in the same manner as the color filter 231.

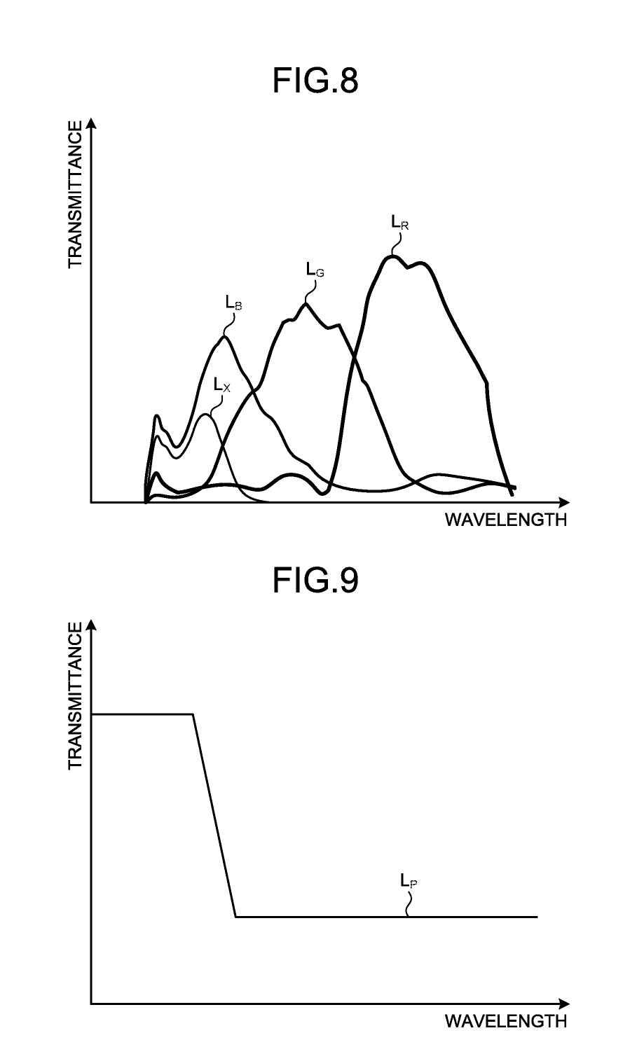

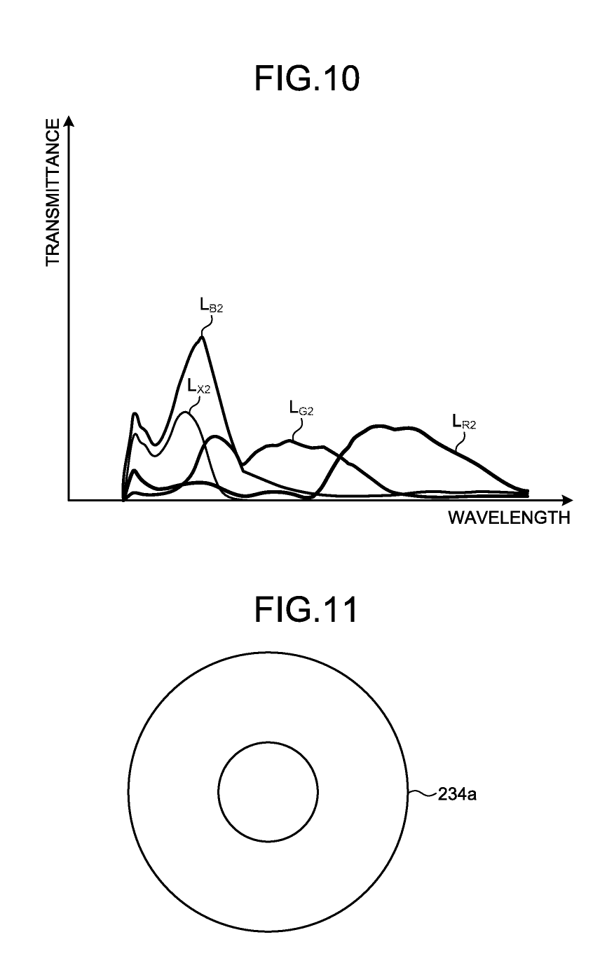

Next, characteristics of the optical filter 234 will be described. FIG. 8 is a diagram illustrating a relationship between transmittance and wavelength of each filter included in the color filter 231. FIG. 9 is a diagram illustrating a relationship between transmittance and wavelength of the optical filter 234. FIG. 10 is a diagram illustrating a relationship between transmittance and wavelength of a combination of the color filter 231 and the optical filter 234. In FIGS. 8 to 10, the horizontal axis represents the wavelength and the vertical axis represents the transmittance. In FIG. 8, a curved line L_{B} represents a relationship between transmittance and wavelength of a wide-band filter B, a curved line L_{G} represents a relationship between transmittance and wavelength of a wide-band filter G, a curved line L_{R} represents a relationship between transmittance and wavelength of a wide-band filter R, and a curved line L_{X} represents a relationship between transmittance and wavelength of a narrow-band filter X. Further, in FIG. 9, a curved line Lp represents a relationship between transmittance and wavelength of the optical filter 234. Furthermore, in FIG. 10, a curved line L_{B2} represents a relationship between transmittance and wavelength of a combination of the wide-band filter B and the optical filter 234, a curved line L_{G2} represents a relationship between transmittance and wavelength of a combination of the wide-band filter G and the optical filter 234, a curved line L_{R2} represents a relationship between transmittance and wavelength of a combination of the wide-band filter R and the optical filter 234, and a curved line L_{X2} represents a relationship between transmittance and wavelength of a combination of the narrow-band filter X and the optical filter 234.

As represented by the curved line L_{X} in FIG. 8, spectral sensitivity of the transmittance of the narrow-band filter X is smaller than that of the curved lines L_{B}, L_{G}, and L_{R} corresponding to the wide-band filters B, G, and R, respectively. Therefore, in the third embodiment, as represented by the curved line L_{P} in FIG. 9, the optical filter 234 that limits light of a predetermined wavelength band, for example, light of a wavelength of 480 nm or more is arranged between the color filter 231 and the light receiving unit 230. Thereby, as represented by the curved lines L_{B2}, L_{G2}, L_{R2}, and L_{X2} in FIG. 10, sensitivity differences between the narrow-band pixel corresponding to the narrow-band filter X and the wide-band pixels corresponding to the wide-band filters B, G, and R, respectively, become small. Thereby, the image sensor 23c can reduce the differences between the amount of light incident on the narrow-band pixel and the amount of light incident on the wide-band pixel, and therefore even when the narrow-band image and the wide-band image are captured at the same time, a high-quality narrow-band image can be acquired.

According to the third embodiment described above, the optical filter 234 equalizes the amount of light incident on the narrow-band pixel with the amount of light incident on the wide-band pixel, and therefore even when the narrow-band image and the wide-band image are captured at the same time, a high-quality narrow-band image can be acquired.

In the third embodiment, the optical filter 234 is arranged between the color filter 231 and the light receiving unit 230. However, the image sensor 23c may be configured by, for example, arranging the optical filter 234, the color filter 231, and the light receiving unit 230 in this order.

### Modified Example of Third Embodiment

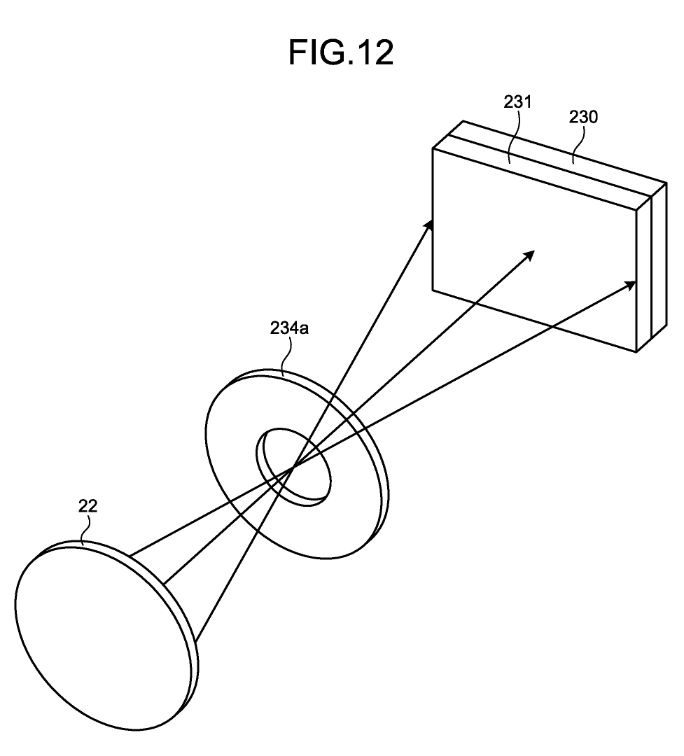

FIG. 11 is a schematic diagram of an optical filter according to a modified example of the third embodiment. FIG. 12 is a diagram schematically illustrating an arrangement of the optical filter according to the modified example of the third embodiment.

As illustrated in FIGS. 11 and 12, an optical filter 234a has an annular shape. The optical filter 234a includes a bandpass filter for passing at least only narrow-band light. The optical filter 234a is arranged between the optical system 22 and the color filter 231. Further, the optical filter 234a is arranged at a pupil position of the optical system 22. With this structure, the image sensor 23c can equalize the amount of light incident on the narrow-band pixel with the amount of light incident on the wide-band pixel, and therefore even when the narrow-band image and the wide-band image, are captured at the same time, each of the narrow-band image and the wide-band image can be acquired with high image quality.

In the modified example of the third embodiment, the optical filter 234a has an annular shape. However, the optical filter may be formed into a disk shape, and a filter for passing wide-band light and narrow-band light may be provided in a center portion of the optical filter. Further, the transmittance of transmitting wavelength may be gradually changed from the center of the optical filter 234a in the radial direction.

### Other Embodiments

In the embodiments described above, a wide-band color filter includes primary color filters. However, complementary color filters (Cy, Mg, and Ye), which transmit light having a complementary color wavelength component, may be used. Further, as a color filter, a color filter (R, G, B, Or, and Cy), which is configured by the primary color filters and filters (Or and Cy) that transmit light having wavelength components of orange and cyan, may be used.

In the embodiments described above, the color filter is provided with a narrow-band filter for passing one type of narrow wavelength band. However, the color filter may be provided with a plurality of types of narrow-band filters. For example, the narrow-band filter X for passing light of a blue wavelength band of 415 nm±30 nm in the first embodiment described above and a narrow-band filter Y for passing light of a green wavelength band of 540 nm±30 nm are provided, and a narrow-band pixel may be generated from an X pixel and a Y pixel.

In the embodiments described above, the imaging device has been described as a capsule endoscope. However, the imaging device may be employed as an endoscope provided at a distal end of an insertion portion that is configured to be inserted into a subject.

According to the some embodiments, even when a narrow-band image and a wide-band image are captured at the same time, it is possible to acquire a high-quality narrow-band image.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An imaging device comprising an image sensor, the image sensor comprising:
a light receiving unit having a plurality of pixels arranged two-dimensionally, the plurality of pixels being configured to receive light from outside and generate an imaging signal in accordance with an amount of the received light;
a color filter having a filter unit disposed corresponding to the plurality of pixels, the filter unit including a plurality of first band filters for passing light of a wavelength band of a primary color or a complementary color and including at least one second band filter for passing narrow-band light whose wavelength band is narrower than the wavelength band of the light passing through each of the plurality of first band filters; and
an output unit configured to output the imaging signal generated by the light receiving unit under conditions that an amount of light incident on a second pixel of the plurality of pixels corresponding to the at least one second band filter is greater than an amount of light incident on each of first pixels of the plurality of pixels corresponding to the plurality of first band filters.

2. The imaging device according to claim 1, wherein
the image sensor further comprises an imaging controller configured to read a first imaging signal from the first pixels corresponding to the plurality of first band filters, and thereafter read a second imaging signal from the second pixel corresponding to the at least one second band filter, and
the output unit is configured to output the first imaging signal and the second imaging signal as the imaging signal.

3. The imaging device according to claim 2, wherein
the imaging controller is configured to set an exposure time of the second pixel corresponding to the at least one second band filter to be longer than an exposure time of each of the first pixels corresponding to the plurality of first band filters to read the first imaging signal and the second imaging signal.

4. The imaging device according to claim 1, wherein
the image sensor further comprises an optical member configured to cause the amount of light incident on each of the first pixels corresponding to the plurality of first band filters to be smaller than the amount of light incident on the second pixel corresponding to the at least one second band filter, and
each of the plurality of pixels is configured to receive the light that has passed through the optical member and the color filter, and generate the imaging signal.

5. The imaging device according to claim 4, wherein
the optical member is an optical filter for passing at least the narrow-band light, and
the optical filter is arranged between the color filter and the plurality of pixels.

6. The imaging device according to claim 4, wherein
the optical member comprises:
a first microlens configured to collect the light onto the first pixels corresponding to the plurality of first band filters; and
a second microlens configured to collect the light onto the second pixel corresponding to the at least one second band filter, wherein
a viewing angle of the second microlens is greater than a viewing angle of the first microlens.

7. The imaging device according to claim 4, wherein
the optical member comprises:
a first light blocking film arranged between the plurality of first band filters and the first pixels and having a first aperture portion with a predetermined size; and
a second light blocking film arranged between the at least one second band filter and the second pixel and having a second aperture portion with an aperture larger than that of the first aperture portion.

8. An endoscope comprising an insertion portion, the insertion portion having the imaging device according to claim 1 at a distal end of the insertion portion.

9. A capsule endoscope comprising:
a capsule-shaped casing configured to be inserted into a subject; and
the imaging device according to claim 1, the imaging device being provided inside the capsule-shaped casing.
